(19) United States
(12) Patent Application Publication
(10) Pub. No.: US 2009/0317838 A1
(43) Pub. Date: Dec. 24, 2009

(54) DETECTION OF BACTERIAL PEPTIDOGLYCAN-LIKE COMPOUNDS

(76) Inventors: Jean-Marc Cavaillion, Chaville (FR); Minou Adib-Conquy, Issy-les-Moulineaux (FR); Oh Yoen KIM, Kyunggi-Do (KR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/405,258

(22) Filed: Mar. 17, 2009

Related U.S. Application Data
(60) Provisional application No. 61/064,633, filed on Mar. 17, 2008.

(51) Int. Cl. G01N 33/569

(52) U.S. Cl. 435/7.32

(57) ABSTRACT

Innate immunity to bacterial pathogens relies on the detection of bacterial peptidoglycans. Intracellular NOD2 proteins sense peptidoglycans and respond by activating transcription factors. An in vitro assay utilizing a reporter gene under the control of a promoter that contains transcription recognition sequences for the binding of a transcription factor that is activated by NOD2 in cells transfected to express NOD2 or NOD2 and TLR2 can identify bacterial infection or sepsis by detecting bacterial peptidoglycans.

### CROSS-REFERENCE TO RELATED APPLICATION

Applicant claims the right to priority based on Provisional Patent Application No. 61/064,633, filed Mar. 17, 2008.

### TECHNICAL FIELD

This invention relates to the pathogen-recognition molecule nucleotide-binding oligomerization domain containing 2 (“NOD2”), which detects bacterial peptidoglycans and induces an anti-bacterial inflammatory response. It also relates to a method of detecting bacterial peptidoglycan-like compounds.

### BACKGROUND ART

Peptidoglycan (“PGN”) components of bacterial cell walls provide bacteria with mechanical protection and confer the characteristic shape of the bacterial cell. Specific to procaryotes, peptidoglycans are present in both Gram-positive and Gram-negative bacterial cell walls, with each Gram type having specific peptidoglycan structural characteristics (Girardin et al., 2003a). The peptide chains form highly cross-liked bridges in Gram-positive bacteria, such as Staphylococcus aureus, and less dense cross-linkages in Gram-negative bacteria, such as Escherichia coli.

Innate immunity to bacterial pathogens relies on the specific sensing of pathogen-associated molecular patterns by pattern-recognition molecules. In mammals, Toll-like receptors (“TLRs”) represent the most extensively studied class of pattern-recognition molecules, which have been shown to sense various molecular patterns, including those found in lipopolysaccharide (“LPS”) and PGN. While TLRs are mainly expressed at the plasma membrane and detect extracellular events, the NOD molecules, a family of intracellular proteins including NOD1/CARD4 and NOD2/CARD15, represent a group of pattern-recognition molecules that sense intracellular bacterial peptidoglycans within the cell cytoplasm. NOD1 and NOD2 both recognize intracellular PGN but respond to different PGN motifs.

The regions of NOD2 responsible for the recognition of PGN motifs have been characterized at the molecular level (Tanabe et al., 2004). Upon PGN binding, NOD1 and NOD2 activate intracellular signal transduction pathways, for example those comprising the transcription factor nuclear factor of kappa light polypeptide gene enhancer in B-cells (“NF-κB”).

Microbial products can be found in the bloodstream in the absence of positive bacteremia, and can contribute to ongoing inflammatory processes, either alone or in synergy with endogenous inflammatory mediators. There exits a need in the art for markers that rapidly and accurately indicate the presence of prokaryotic organisms, e.g., bacterial infection or translocation. Surgery, for example, vascular surgery, is associated with an inflammatory process and an alteration of the immune status. Abdominal aortic aneurysm surgery is thought to be associated with bacterial translocation through the gut barrier, during manipulation of the gut by the surgeon, and aortic clamping. The significant decrease in mesenteric blood flow and the subsequent alteration of oxygen delivery to the intestinal epithelial carriers has been proposed as a mechanism for the translocation.

Markers of the invention would find use, inter alia, in monitoring at risk patients, for example in monitoring the occurrence of nosocomial infections among intensive care unit patients. Currently available markers include myeloid surface markers and procalcitonin, but they are limited in their ability to distinguish acute inflammation from bacterial presence (Adib-Conquy et al., 2007). For example, one marker, procalcitonin, “cannot reliably differentiate sepsis from other non-infectious causes of systemic inflammatory response syndrome” (Tang et al., 2007).

The detection of microbial-derived products provides an alternative, but, to date, an unsatisfactory approach to developing infection-specific markers. For example, endotoxin, or LPS, can be detected in sepsis, but is also present in numerous other non-infectious clinical settings, as it can translocate from the intestine following successful cardiopulmonary resuscitation after cardiac arrest, cardiopulmonary bypass surgery, hemorrhagic shock, liver transplantation, cirrhosis, thermal injury, lethal irradiation and bone marrow transplant, and pancreatitis (Adrie et al., 2002; Cabiè et al., 1993; Balzan et al., 2007). The silkworm larvae test, which can detect PGN, lacks specificity for bacteria, as it also detects fungal components (Shimizu et al., 2005). Additionally, measuring endotoxin in the plasma poses difficulties due to the presence of interacting molecules, such as soluble CD 14, LPS-binding protein, and high density lipoprotein. Furthermore, endotoxin is only derived from Gram-negative bacteria; thus, it cannot provide a marker for Gram-positive infections.

### SUMMARY

The present invention provides a method of detecting bacterial infection, e.g., sepsis, and/or bacterial translocation, by measuring changes in the luciferase activity of cells transfected with NOD2 or NOD2 and TLR2 and an NF-κB-luciferase gene reporter, in samples comprising PGN. In certain embodiments, methods of the invention can detect all types of bacteria in biological fluids. NOD2 senses the minimal PGN motif muramyl dipeptide (“MDP”), which is present in the PGN of both Gram-positive and Gram-negative bacteria.

The invention provides a human embryonic kidney (HEK) cell line transfected with two plasmids, one allowing the constitutive expression of NOD2 or NOD2 and TLR2 and one the expression of the luciferase gene, under the control of a promoter, which contains transcription recognition sequences for the binding of a transcription factor activated by NOD2, e.g., an NF-κB-dependent promoter. NOD2 is involved in sensing the presence of PGN or fragments of PGN, and initiates an intracellular cascade that leads to NF-κB activation and luciferase expression.

In an aspect, this invention provides a method of detecting a peptidoglycan or a peptidoglycan-like compound in a sample by transfecting a cell with a vector comprising a sequence encoding NOD2, co-transfecting that cell with a vector comprising a nucleotide sequence encoding a reporter protein, placing the cell in contact with the sample, and measuring the reporter gene expression in the cell, wherein the reporter gene expression indicates the presence of peptidoglycan or peptidoglycan-like compound in the sample. Cells may be transiently or stably transfected, using any method known in the art.

In an embodiment, the peptidoglycan is detected by measuring the activation of a transcription factor activated by NOD2, e.g., NF-κB. In another embodiment, the reporter gene is under the control of a promoter, which contains transcription recognition sequences for the binding of a transcription factor activated by NOD2. In a further embodiment, the vector comprising the sequence encoding the reporter protein further comprises an enhancer region of the transcription factor. In yet a further embodiment, the promoter controls NF-κB. In another embodiment, the reporter gene is detected by a biolumninescent signal.

NOD2 activates transcription factors and activates signaling cascades. These factors and signaling pathways are known in the art. (See, e.g., Park et al., 2007 and Voss et al., 2006.) Accordingly, the invention provides methods of detecting peptidoglycan by measuring reporter gene function of NOD2 activated transcription factors and signaling cascades.

The invention provides a method of detecting PGN or PGN-like compounds in a sample, e.g., plasma. In an embodiment, the sample is taken from a patient with sepsis. In another embodiment, the sample is taken from a surgical patient, either prior to, during, or after surgery, e.g., abdominal surgery, including abdominal aortic surgery. Samples can come from humans or other animals. They can also come from water, food, and any substance suspected of bacterial contamination.

In another aspect, the invention provides a method for detecting the presence or absence of a bacterial infection in a sample, wherein the method consists essentially of providing a sample from a subject, providing a cell having an intact cell wall, and wherein the cell expresses NOD2, and providing a reporter gene; contacting the cell and the sample by extracellular contact of the sample with the intact wall of the cell and without injecting the sample into the cell; incubating that cell under conditions which maintain its viability; determining whether the reporter gene is expressed; and correlating expression of the reporter gene with the presence or absence of the bacterial infection.

In an embodiment, the presence of the bacterial infection is detected by measuring the activation of a transcription factor activated by NOD2, e.g., NF-KB. In another embodiment, the reporter gene is under the control of a promoter, which contains transcription recognition sequences for the binding of a transcription factor activated by NOD2. In a further embodiment, the vector comprising the sequence encoding the reporter protein further comprises an enhancer region of the transcription factor. In yet a further embodiment, the promoter controls NF-KB. In another embodiment, the reporter gene is detected by a biolumninescent signal.

In an embodiment, the extracellular contact occurs in the absence of cell transfection reagents. In another embodiment, the extracellular contact occurs in the absence of lipid emulsifying or solubilizing compounds.

In an embodiment, the expression of the reporter gene is compared with expression of a reporter gene in a cell that expresses fsNOD2 and a promoter that contains transcription recognition sequences for the binding of a NOD2-activated transcription factor linked to a reporter gene as a control. In another embodiment, the sample is plasma, e.g., from a patient undergoing surgery or a patient with sepsis. In an embodiment, the cell is a human embryonic kidney cell and the reporter is luciferase.

In another aspect, the invention provides a kit for practicing a method of detecting a PGN or PGN-like compound in a sample by transfecting a cell with a vector comprising a nucleotide sequence encoding NOD2, co-transfecting that cell with a vector comprising a nucleotide sequence encoding a reporter protein, placing the cell in contact with the sample, and measuring the reporter gene expression in the cell, wherein the reporter gene expression indicates the presence of PGN or PGN-like compounds in the sample. In one embodiment, kits of the invention comprise a vector comprising a sequence coding for NOD2, a vector comprising a sequence coding for a reporter protein, cells to be transfected with these vectors, and instructions for use. Optionally, the reporter protein is under the control of a promoter that contains transcription recognition sequences for the binding of a transcription factor that is activated by NOD2.

In a preferred embodiment of this invention, the sensitivity of PGN detection is increased. This is achieved by using both NOD2 and TLR2 with the reporter.

### BRIEF DESCRIPTION OF THE DRAWINGS

FIG. 1: Experimental Procedure. Cultured HEK293T cells were co-transfected with a vector comprising a nucleotide sequence encoding NOD2 and the reporter plasmid pNF-κB-Luc, then stimulated with plasma containing PGN to induce luciferase expression.

FIG. 2: MDP or PGN Activates NF-κB. Muramyl dipeptide (“MDP”) (FIG. 2A) and peptidoglycan (“PGN”) (FIG. 2B) induce luciferase expression in HEK293T cells co-transfected with a vector comprising a nucleotide sequence encoding NOD2 and pNF-κB-Luc plasmid.

FIG. 3: NOD2 Detects PGN From Gram-Positive and Gram-Negative Bacteria. PGN from Gram-positive Staphylococcus aureus bacteria and from Gram-negative Escherichia coli bacteria induce luciferase expression in HEK293T cells co-transfected with a vector comprising a nucleotide sequence encoding NOD2 and pNF-κB-Luc, when added to human plasma.

FIG. 4: A Frameshifted NOD2 Mutant Does Not Activate NF-κB in Plasma from Healthy Donors. Cells co-transfected with pNF-κB-Luc and a NOD2 plasmid with the 3020InsC frameshift mutation have lost the ability to respond to MDP; but remain responsive to the inflammatory cytokines tumor necrosis factor-α (“TNF”) and interleukin 1β (“IL-1β”).

FIG. 5: Plasma From Septic Patients Activates NF-κB. Plasma from patients with sepsis, but not from healthy donors, induce luciferase expression in HEK293T cells co-transfected with a vector comprising a nucleotide sequence encoding NOD2 and pNF-κB-Luc.

FIG. 6: A Frameshifted NOD2 Mutant Does Not Activate NF-κB in Plasma from Septic Patients. Cells transfected with pNF-κB-Luc and co-transfected with a vector comprising a nucleotide sequence encoding NOD2, but not those co-transfected with NOD2 3020InsC, respond to plasma from septic patients.

FIG. 7: Bacterial Translocation in Patients Undergoing Abdominal Aortic Surgery. Plasma PGN levels increase in patients undergoing abdominal surgery, but not carotid surgery, which does not involve abdominal manipulation, and thus does not induce bacterial translocation.

FIG. 8: A depiction of a scheme to increase the sensitivity of PGN detection using anaerobic bacterial PGN and cells co-transfected with NOD2 and TLR2 or NOD1. Modified from Brian Kelsall, Getting to the guts of NOD2. Nature Medicine 11, 383-394 (2005); Warren Strober, Peter J. Murray, Atsushi Kutani & Tomohiro Watanabe, Signaling pathways and molecular interactions of NOD1 and NOD2, Nature Reviews Immunology 6, 9-20 (2006); and C. Werts, S E Girardin. D J Philpott, TIR, CARD and PYRIN: three domains for an antimicrobial triad, Cell Death and Differentiation (2006) 13, 798-815 (2006).

FIG. 9: Sensitivity of PGN detection is increased using NOD2 and TLR2.

FIG. 10: Sensitivity of PGN detection was not increased using NOD2 and NOD1.

FIG. 11: Detection of circulating peptidoglycan using NOD2 transfected cell line and NF-κB-luciferase reporter gene. (A) Activation with muramyl dipeptide (MDP) or peptidoglycan (PGN) from Staphylococcus aureus (S. aureus). Human embryonic kidney (HEK) 293T cells transfected with NOD2 and NF-κB luciferase expression plasmids were stimulated with MDP or S. aureus PGN. After 6 hours of incubation, luciferase activity in cell extracts was measured and expressed as relative light unit (RLU). The Figure is the mean±SEM of three independent experiments performed in triplicates. (B) Sensitivity of NOD2-detection of both Gram-negative and Gram-positive bacterial PGN added in healthy human plasma. HEK293T cells transfected with NOD2 and NF-κB luciferase expression plasmids were stimulated with various concentrations of PGN from S. aureus or Escherichia coli diluted in plasma from healthy controls. The Figure is the mean±SEM of three independent experiments performed in triplicates. (C) NOD2-detection of both purified and non-purified anaerobic Gram-negative and Gram-positive bacterial PGN incubated in healthy human plasma. HEK293T cells transfected with NOD2 and NF-κB luciferase expression plasmids were stimulated with purified or non-purified anaerobic bacterial PGN from Gram-positive (Clostridium clostridioforme) and Gram-negative (Bacteroides thetaitamicron) diluted in plasma from healthy controls. The Figure is the mean±SEM of three independent experiments performed in triplicates. (D) Positive signals in plasma from septic patients. HEK293T cells transfected with NOD2 and NF-κB luciferase expression plasmids were stimulated with plasma from healthy controls and sepsis patients. As a positive control, MPD was used. The Figure is the mean±SEM of three independent experiments performed in triplicates. (E) Specificity of NOD2 detection of bacterial PNG fragment in comparison with a frame shift mutant of NOD2 (fsNOD2) transfected system. HEK293T cells transfected with NOD2 or fsNOD2 and NF-κB luciferase expression plasmids were stimulated with MDP, tumor necrosis factor-α (TNF-α), or interleukin-1β (IL-1β). The Figure is the mean±SEM of three independent experiments performed in triplicates. (F) Comparison of the signals between NOD2 and fsNOD2 in septic plasma. HEK293T cells transfected with NOD2 or fsNOD2 and NF-κB luciferase expression plasmids were stimulated with plasma samples from sepsis patients. The Figure is the mean±SEM of three independent experiments performed in triplicates.

FIG. 12: Assessment of bacterial peptidoglycan levels in the plasma of AAS and CAS patients. HEK293T cells transfected with NOD2 and NF-κB luciferase expression plasmids were stimulated with plasma from AAS and CAS patients. After 6 hours of incubation, luciferase activity in cell extracts was measured, and NF-κB activation was calculated as fold induction with respect to time point 1 (T₁). T₁ (before anesthesia), T₂ (before incision), T₃ (before clamping), T₄ (after blood reperfusion), POD1 (postoperative day 1), POD2 (postoperative day 2). Data are shown as median and interquartile. The two groups were compared using ANOVA repeated measurement and least significant difference. P-value (p=0.003) indicates the significant difference between the two patient groups in whole study time. *P<0.05 and **P<0.01 are from the comparison of the two groups at each time point (Mann-Whitney U test). CAS: carotid artery surgery (white boxes), AAS: abdominal aortic surgery (grey boxes). Black and white circles (T₂ to POD2) indicate values within 5^{th} percentile and out of 95^{th} percentile, respectively.

FIG. 13: Assessment of endotoxin (LPS) associated with circulating peripheral blood mononuclear cells (PBMC) in AAS and CAS patients. Endotoxin associated with patients' PBMC was measured using a Limulus amebocyte assay. T₁ (before anesthesia), T₂ (before incision), T₃ (before clamping), T₄ (after blood reperfusion), POD1 (postoperative day 1), POD2 (postoperative day 2). Data are shown as median and interquartile. The two groups were compared using ANOVA repeated measurement and least significant difference. P-value (P=0.006) indicates the significant difference between the two patient groups in whole time measured. *P<0.05 are from the comparison of the two groups at each time point (Mann-Whitney U test). CAS: carotid artery surgery (white boxes), AAS: abdominal aortic surgery (grey boxes). Black and white circles (T₁ to POD2) indicate values within 5^{th} percentile and out of 95^{th} percentile, respectively.

FIG. 14: Comparison of the detection tests for circulating PGN in plasma, and for circulating and PBMC-associated endotoxin. The Figure indicates the percentage (%) of positive patients based on the detection limits. The detection limit of PGN levels was 1.57 fold increase with respect to the levels before anesthesia (T₁). It was calculated based on the mean±2 standard deviations of the fold values at T₂ in control subjects. Detection limit of endotoxin level associated with PBMC was 5.0 pg/ml, that measured in plasma was 1.6 pg/ml. The comparison was performed at the peak for each marker. CAS: carotid artery surgery, AAS: abdominal aortic surgery. ND: Not done

### DETAILED DESCRIPTION

### Definitions

The terms used herein have their ordinary meanings or the meanings as set forth below, and can be further understood in the context of the specification.

A “polysaccharide” is a carbohydrate polymer of monosaccharides. It may comprise starch and/or sugar monosaccharides and may be linear or branched. A polysaccharide may further comprise lipid and thus be referred to as a lipopolysaccharide (“LPS”).

A “peptidoglycan” is a polysaccharide covalently linked to short peptides, arranged as parallel strands of polysaccharide, having a glycan backbone of N-acetylglucosamine (“GlcNAc”) and N-acetyl muramic acid (“MurNAc”) arranged in β1,4 linkages. The polysaccharide is cross-linked with covalently bound short peptides comprising l-alanine and one or more d-amino acids, e.g., d-glutamic acid, d-alanine, and d-diaminopimelic acid. As used herein, the term “peptidoglycan” encompasses peptidoglycans and peptidoglycan-like compounds.

“Peptidoglycan-like compounds” are peptidoglycan degradation products, e.g., PGN fragments derived from bacteria. They can be derived from dead bacteria and can be released during bacterial division.

A “protein” is a polymeric form of amino acids of any length. The terms “peptide” and “polypeptide” may be used interchangeably. “Proteins” include naturally-occurring amino acids, coded and non-coded amino acids, chemically modified amino acids, amino acid analogs, and peptidomimetics. A “lipoprotein” is a protein conjugated with a lipid.

A “sample” is any portion of an entity to be tested, which is generally representative of the entity. Samples can be solid or liquid, include food and water, and can comprise any substance suspected of having bacterial contamination.

A “biological sample” is a portion of any biological organism. The term includes, for example, biological fluids such as blood, serum, plasma, urine, cerebrospinal fluid, tears, saliva, lymph, dialysis fluid, lavage fluid, semen, and other liquid samples of biological origin. A biological sample can include cells and their progeny, including cells in situ, cells ex vivo, cells in culture, cell supernatants, and cell lysates. It can include organs, tissues, tissue biopsy samples, tumor biopsy samples, stool samples, fluids extracted from cells and tissues, and tissue culture-derived fluids. Cells dissociated from solid tissues, tissue sections, and cell lysates are also included. A biological sample can comprise a sample that has been manipulated after its procurement, such as by treatment with reagents, solubilization, or enrichment for certain components, such as polynucleotides, polypeptides, lipids, or polysaccharides.

“NOD1” refers to nucleotide-binding oligomerization domain containing 1, a molecule which serves as an intracellular receptor for bacterial peptidoglycans and is involved in signal transduction leading to NF-κB activation. It is also known in the literature as caspase recruitment domain-containing protein 4 (“CARD4”). The human genomic reference sequences are National Center for Bioinformation Technology (“NCBI”) NC_{—}000007.12, NT_{—}007819.16, and NT_{—}079592.2. The UniProt/SwissProt reference number to human NOD1 is Q9Y239. The full-length NOD1 protein has an amino terminal caspase recruitment domain, a nucleoside triphosphatase domain, and nine terminal leucine-rich regions. The caspase recruitment domain activates NF-κB pathways and the leucine-rich regions mediate the recognition of bacterial components.

The term “NOD1” encompasses nucleic acids and peptides. It encompasses all known variants, including isoforms, splice variants, and single nucleotide polymorphisms. The term encompasses functional mutants, and fragments, having either the same, an increase, or a decrease in function, including, but not limited to, the functions of transcription factor activation and peptidoglycan binding. It also encompasses orthologs from non-human species, including dog, chimpanzee, rat, mouse, and chicken.

“NOD2” refers to nucleotide-binding oligomerization domain containing 2, a molecule which, like NOD1, serves as an intracellular receptor for bacterial peptidoglycans and is involved in signal transduction leading to NF-κB activation. It is also known in the literature as caspase recruitment domain-containing protein 15 (“CARD15”). The human genomic reference sequences are NCBI NC_{—}000016 and NT_{—}010498. The UniProt/SwissProt primary reference number to human NOD2 is Q9HC29 and the secondary accession numbers are Q96RH5, Q96RH6, and Q96RH8. The full-length NOD2 protein has two amino terminal caspase recruitment domains, a nucleotide-binding domain, and ten carboxyl terminal leucine-rich region. The caspase recruitment domains activate NF-κB pathways and the leucine-rich regions mediate the recognition of bacterial components.

The term “NOD 2” encompasses nucleic acids and peptides. It encompasses all known variants, including isoforms, e.g., Q9HC29-1 and Q9HC29-2. It encompasses splice variants and single nucleotide polymorphisms. The term encompasses functional mutants, and fragments, having either the same, an increase, or a decrease in function, including, but not limited to, the functions of transcription factor activation and peptidoglycan binding. It also encompasses orthologs from non-human species, including dog, chimpanzee, rat, mouse, and zebrafish.

A “reporter gene” is a gene comprising a detectable reporter molecule under the control of regulatory elements of a gene of interest. The regulatory sequences control the conditions under which the gene of interest can be expressed, and the reporter molecule provides information about gene expression.

“NF-kB” is a transcription factor that controls the expression of multiple genes. Signal transduction pathways that utilize NF-κB-regulated transcription generally regulate genes encoding proteins involved in immune and inflammatory responses and with cell growth control.

“Gram-positive” and “Gram-negative” bacteria, are bacteria which stain either purple or pink, respectively, following a Gram stain. The Gram stain generally comprises fixing the bacterial cell, staining it with crystal violet, treating with iodine, decolorizing with alcohol, and counterstaining. Gram-positive refers to the ability of a bacterium to resist decolorization with alcohol following the crystal violet stain, imparting a purple color to the bacterium when viewed by light microscopy. Gram-negative refers to the susceptibility to the decolorization and counterstaining procedures.

Gram-positive bacteria typically have a relatively thick cell wall, made up largely of peptidoglycan. The peptidoglycan layer in Gram-negative bacteria is thin and surrounded by an outer membrane. Both Gram-positive and Gram-negative bacteria take up the crystal violet and iodine stains. The crystal violet and iodine complex becomes trapped inside the Gram-positive cell by the dehydration step, which reduces the porosity of the thick cell wall. The thin peptidoglycan layer does not impede the extraction of the crystal violet and iodine complex from the Gram-negative cell.

“Viability” refers to the ability to adequately live, function, or develop, under favorable conditions.

“Bacterial infection” refers to the invasion by and multiplication of pathogenic bacteria in any body tissue or fluid. It may be clinically apparent or inapparent; local or systemic; and acute, subacute, or chronic.

“Sepsis” refers to a systemic infection and includes the presence of bacteria or other infectious organisms, or components or products of these organisms, including, but not limited to, PGN and toxins, in the blood or other tissues.

The terms “patient” and “subject” are used interchangeable herein, and refer to an animal, preferably, a mammalian human or non-human animal.

A “surgical patient” is a patient who has undergone, is currently undergoing, or is a candidate for undergoing surgery.

“Injecting” refers to the introduction of a substance by force into a body part.

Immunity to Bacterial Pathogens

Innate immunity to bacterial pathogens relies, in part, on the intracellular detection of bacteria that have gained entry into the cell. The NOD family, including NOD1 and NOD2, sense bacterial products within the cytoplasm and allow detection of intracellular invasive bacteria. NOD1 and NOD2 both detect peptidoglycans but recognize different molecular motifs within the peptidoglycans (Girardin et al., 2003a). The PGN motif sensed by NOD2, MDP, is found in all bacteria, thus NOD2 is a general sensor of PGNs and their degradation products (Girardin et al., 2003b).

Upon sensing peptidoglycans, NOD1 and NOD2 activate NF-κB, a nuclear transcription factor that regulates the expression of numerous immune system components. Activation of the NF-κB pathway in turn activates pro-inflammatory proteins, contributing to the generation of an immune response. Stimulation of immune responses in a wide variety of mammalian cells leads to NF-κB activation. Many different intracellular signals can induce NF-κB activation, for example, bacterial or viral infection, hormones, stress, UV irradiation, B or T-cell activation, LPS, and certain cytokines, e.g., TNF or IL-1. In resting cells, NF-κB is typically found in the cytoplasm and translocates to the nucleus in response to an extracellular signal, where it binds to specific DNA sites to regulate transcription.

Reporter Assays for NF-κB Activation

Reporter assays generally employ vectors having a reporter gene downstream of a cloning site. The reporter gene is typically chosen to be a protein that is not found in humans and is simple to assess for a readout signal. Classes of reporter genes suitable for use in detecting NOD2-dependent transcription include bioluminescent, chemiluminescent, radioactive, and fluorescent molecules. Reporter genes are known in the art and commercially available in many forms. Those commonly used to examine the control of eucaryotic gene expression include firefly luciferase, which encodes a gene product that catalyses the reaction between luciferin and ATP, producing photons of light detectable in a chemiluminescent bioassay for ATP. Other reporter genes suitable for use in the invention include, but are not limited to, the bacterial chloramphenicol acetyl transferase gene and β-galactosidase, the product of the lacZ gene, which encodes an enzyme that hydrolyses the beta galactoside linkage in lactose to produce glucose and galactose. β-galactosidase also hydrolyses the chromogenic substrate isopropylthiogalactoside. Additional suitable reporter genes include alkaline phosphatase, which catalyses the cleavage of inorganic phosphate non-specifically from a wide variety of phosphate esters, and green fluorescent protein (GFP), a jellyfish protein that fluoresces with green visible light when excited with ultraviolet light. GFP can be humanized such that codons of the naturally-occurring nucleotide sequence are changed to more closely match the human codon bias.

Reporter vectors of the invention can be used to monitor the activation of the signal transduction pathways converging at a transcription factor activated by NOD2, such as NF-κB response elements. Reporter vectors are well-known in the art, can be made using conventional methods, and can be purchased from commercial sources. The activation of endogenous signal transduction pathways initiated by extracellular stimuli and mediated by a co-transfected gene will result in the activation of corresponding trans-activators, which, in turn, stimulate reporter expression.

The reporter systems of the invention can provide information regarding the components, function, and effects of cellular signal transduction pathways. In particular, they can be used to detect components of the pathways. In vivo, extracellular signals, e.g., bacterial peptidoglycans, trigger the activation of a series of intracellular signaling molecules, which form a signal transduction pathways converging at transcription factors. The activation of the signal transduction pathway can be monitored by the expression level of a reporter gene controlled by a synthetic promoter containing enhancer elements specific to that transcription factor.

Transfection methods are well-known in the art. Cells of the invention may be transfected by any known method, including calcium phosphate transfection, electroporation, and lipid-mediated methods. Adherent cells, which can be transfected by conventional means, are suitable for use in the invention. In one embodiment, cells are stably transfected, and in another embodiment, the cells are transiently transfected. In an embodiment, transfection is performed while the cells are plated in wells of cell culture plates.

Any cells known in the art to be capable of transient or stable transfection are suitable for use in the invention, for example HEK293T, Hela, Raw 264.7, and THP-1 cells. Candidates for transfection by the vectors of the invention either do not express, or express low levels of NOD1, NOD2, TLR2, and/or TLR4.

The reporter assay can detect the presence or absence of a bacterial infection in a biological specimen, e.g., plasma, in untransfected cells. Cells co-expressing NOD2 and a reporter gene under the control of a promoter that contains transcription recognition sequences for the binding of a transcription factor, which is activated by NOD2, but expressing little or no NOD1, are placed in contact with the specimen. Suitable cells include, but are not limited to, human embryonic kidney cells. The specimen contacts the extracellular surface of the cells without gaining entry to the cell, i.e., without breaching the membrane or otherwise entering the cell, e.g., by emulsifying with or solubilizing into the lipid bilayer. This reporter assay can detect the presence or absence of bacterial infection from a sample comprising substances other than NOD2 ligands, e.g., the sample may comprise components of mammalian or bacterial cells, including proteins, lipids, and polysaccharides. It can identify activators of the NOD2 pathway in samples which also contain such components, including cellular debris and endogenous enzymes, which induce cell autolysis.

Assays of the invention using transfected cells can be used to detect bacterial translocation in patients undergoing surgery. Frameshift mutations can be induced in the plasmids of the invention using methods known in the art.

Cells can be co-transfected with both NOD1 and NOD2. Cells can also be co-transfected with both NOD2 and toll-like receptor 2 (“TLR2”), or with both NOD1 and TLR2. TLR2 (GenBank™/EBI accession number 060603), also known as the CD282 antigen, plays a role in mediating the innate immune response to bacterial lipoproteins through the NF-κB pathway. TLR2 has been shown to be implicated in PGN detection but does not, in the absence of cofactors, activate the NF-κB pathway (Girardin et al., 2003b). The literature provides conflicting reports regarding the role of TLR2 in PGN detection. Dziarski et al. have demonstrated that highly purified PGN activates the TLR2 signaling pathway. Travassos et al, however, attributes the apparent response of TLR2 to PGN to lipoprotein contamination. Regardless, in a natural setting, highly purified molecules do not exist, and PGN activates cells through TLR2.

The reporter assay detects anaerobic bacterial PGNs. Anaerobic bacteria are present in the intestine and likely to undergo translocation during surgery. Reporter assays of the invention can comprise genes coding or molecules that transport PGN into the cell's cytoplasmic compartment. With the exception of invasive bacteria, e.g., Shigella or Listeria, adaptor and/or transporter proteins internalize PGN. For example, hPepT1 human peptide transporter 1, a transmembrane transporter of di- or tri-peptides, has been shown to internalize MDP.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges including either or both of the upper and lower limits of the range, unless specifically excluded from the stated range.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Reporter Assay for NF-κB Activation

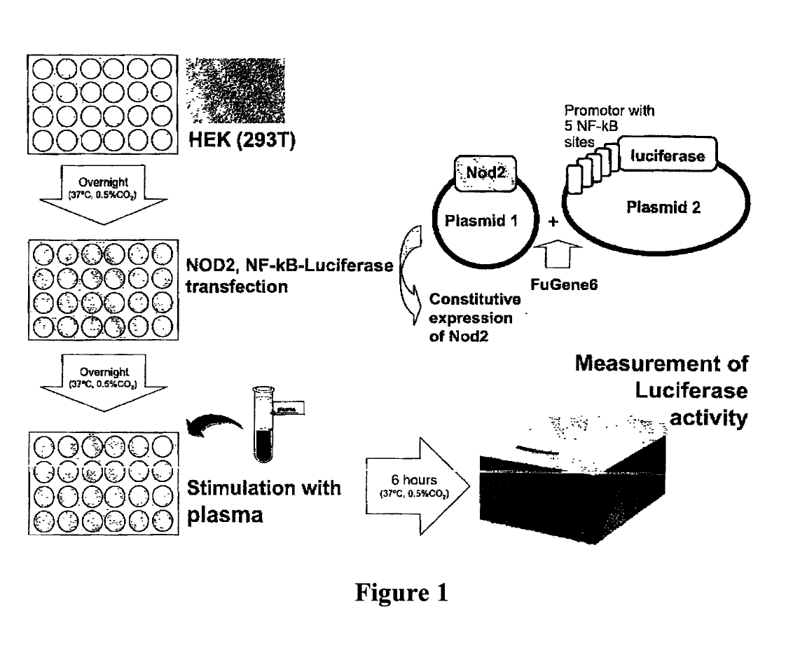

Human embryonic kidney HEK293T cells (American Type Culture Collection (“ATCC,”) Manassas, Va., USA) were cultured in Dulbecco's modified Eagle's medium (Invitrogen, Carlsbad, Calif., USA) supplemented with 10% fetal calf serum (“FCS”) (PAA Laboratories GmbH, Pasching, Austria). HEK293T cells were seeded into 24 well cell culture plates at a density of 10⁵ cells/ml (500 μl/well) and transfected with various expression plasmids, as previously described (Girardin 2003a) (FIG. 1).

Transfection was performed as described by Girardin et al., 2003a. HEK293T cells were transfected overnight using 1% FuGENE 6 transfection reagent (Roche Diagnostics, Mannheim, Germany) with 75 ng of the reporter plasmid pNF-kB-Luc (Stratagene, La Jolla, Calif. USA) and 1 ng p-UNO-hNODt (InvivoGen, San Diego, Calif. USA) or fsNOD2 expression plasmids (Begue et al. (2006); Chamaillard et al. (2003)). The plasmid pNF-κB-Luc is a 5.7 kb cis-reporting pNF-κB-Luc plasmid comprising an NF-κB enhancer region and a firefly luciferase gene controlled by a synthetic promoter that contains direct repeats of the transcription recognition sequences for the binding sites for NF-κB (Stratagene, Carlsbad, Calif., USA). The NOD2 3020InsC frameshift mutation was introduced using the QuikChange XL site-directed mutagenesis kit (Stratagene, Carlsbad, Calif., USA). The entire coding region was verified by sequencing and the size of the encoded product was verified by immunoblotting (Girardin et al., 2003b). A total plasmid concentration of 250 ng was achieved by addition of pcDNA3.1 vector.

Following a twenty hour transfection period, 50 μl of plasma was added into the wells containing the transfected cells. Purified peptidoglycan, diluted in plasma, from S. aureus (Sigma, St. Louis, Mo., USA) and E. coli (kind gift of Dominique Mengin-Lecreulx or InviroGen, San Diego, Calif., USA) served as positive controls. After six hours of peptidoglycan stimulation, the cell supernatant was removed and the cells were lysed with 100 μl of lysis buffer (25 mM Tris-phosphate pH 8, 8 mM MgCl₂, 1 mM dithiothreitol, 15% glycerol, and 1% Triton X-100). Luciferase expression was measured from a 10 μl aliquot of cell extract using a microplate luminometer (LB960 luminometer centro, Berthold Technologies, Bad Wildbad, Germany) after addition of 100 μl of substrate buffer to a final concentration of 1.8 mM luciferin and 1 mM ATP. Each measurement was performed in triplicate. The detection level of the assay is at least 0.15 ug/ml PGN, determined by adding purified PGN to healthy donor plasma.

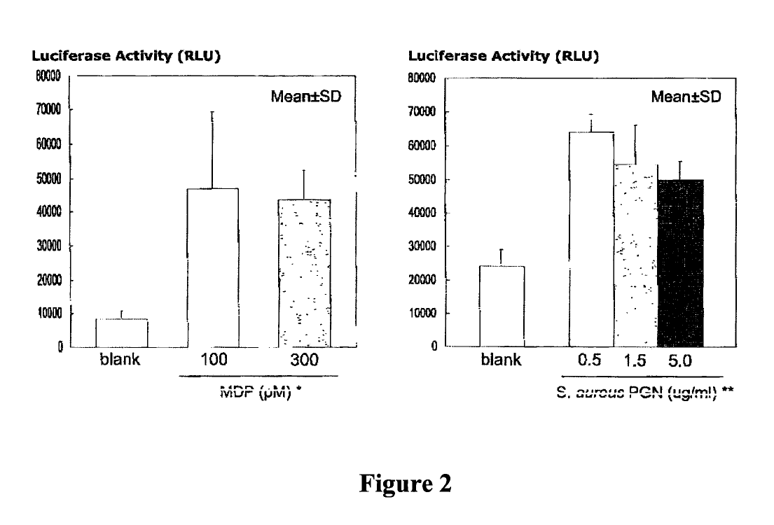

To validate the experimental system, NF-κB activation in HEK293T cells co-transfected with pNF-κB-Luc and pUNO-hNOD2 was tested in response to stimulation with both MPD and PGN derived from S. aureus (FIG. 2). NF-κB activity was expressed in terms of relative light units (RLU). NOD2 senses the presence of both PGN and fragments of PGN, e.g., MDP, and initiates an intracellular response that leads to NF-κB activation and subsequent luciferase transcription and expression. Thus, this system can detect PGN and MDP, the smallest active PGN NOD2 ligand. MDP was added simultaneously with FuGENE 6 to induce its entry into the cytoplasm, while PGN entered the cytoplasmic compartment regardless of whether it was added with the transfection reagent or at a later time.

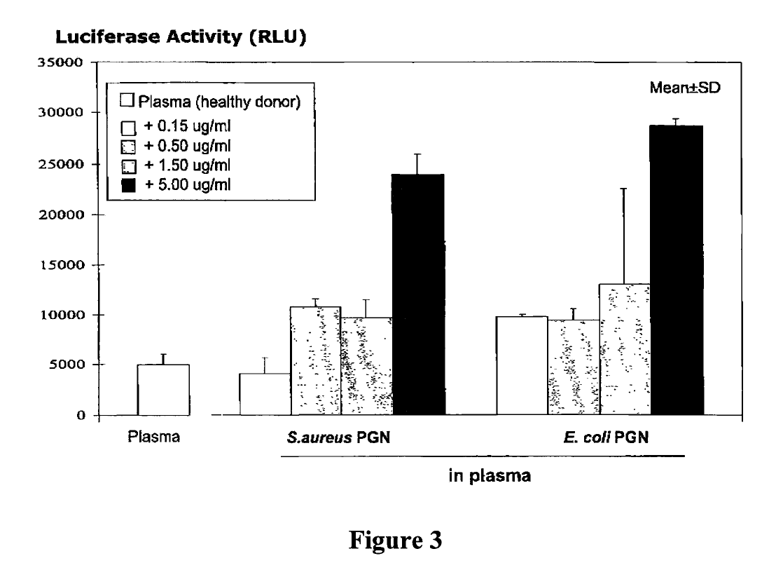

As another positive assay control, PGN from Gram-positive S. aureus and Gram-negative E. coli were diluted in plasma from healthy control subjects and added to HEK293T cells co-transfected with pNF-kB-Luc and pUNO-hNOD2. PGN from both bacterial sources increased luciferase expression in a generally dose-dependent manner (FIG. 3).

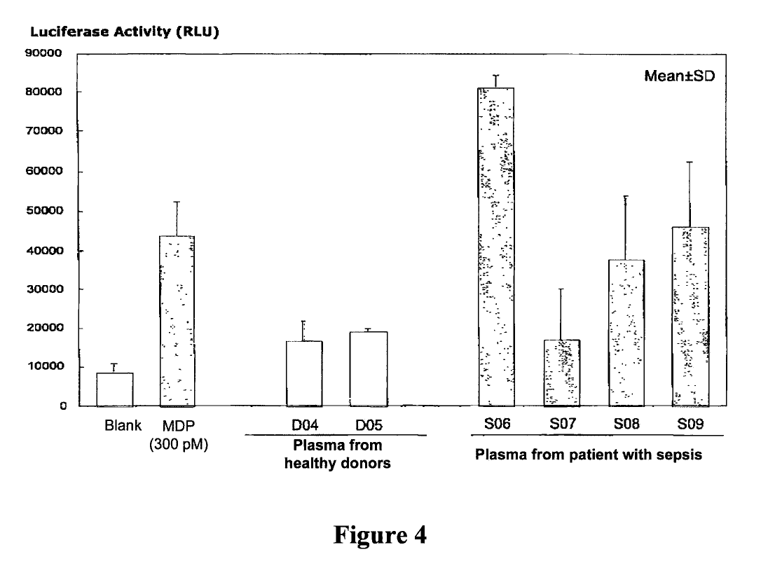

The specificity of the NF-κB activation assay was demonstrated by co-transfecting HEK293T cells with pNF-κB-Luc and a frameshift mutant of NOD2. The NOD2 3020InsC variant comprises a Leu1007-Pro substitution in the tenth leucine-rich region followed by a premature stop codon. It activates the NF-κB pathway to the same extent as wild-type NOD2 when overexpressed, but is unable to detect MDP or PGN (Girardin et al., 2003b). Cells transfected with the NOD2 plasmid comprising the frameshift mutation demonstrated no increase in luciferase in response to MDP (FIG. 4). This indicates that the assay is specific for NOD2 detection of peptidoglycans. The assay responds to the inflammatory mediators TNF and IL-1β, which activate endogenous receptors in signal transduction pathways that activate NF-κB, further demonstrating its specificity.

### Example 2

### NF-κB Activation Stimulated with Plasma from Septic Patients

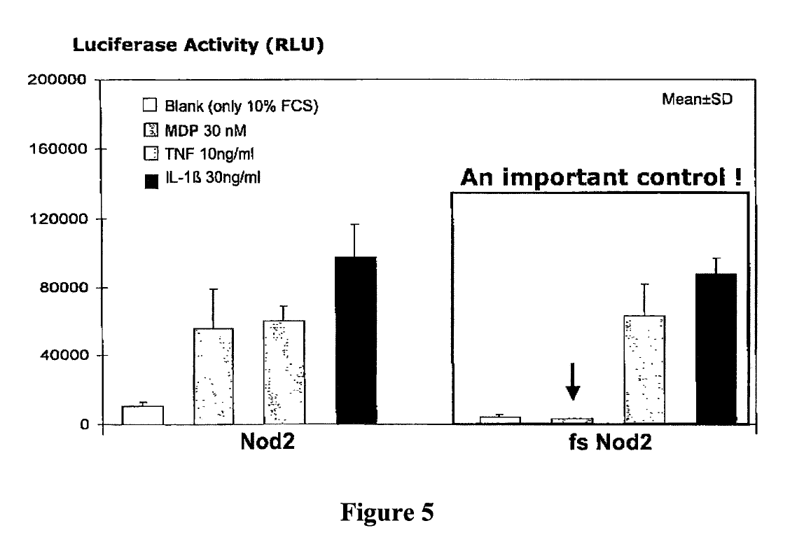

HEK293T cells co-transfected with the pNF-κB-Luc and pUNO-hNOD2 plasmids were assayed for luciferase expression in the presence of plasma from healthy donors and plasma from donors suffering from sepsis. FIG. 5 shows that 300 pM MDP induced NF-κB activation. Plasma samples from four patients with sepsis, S06-S09, were compared to plasma samples from two healthy donors, D04-D05. Three of the four septic samples, but neither of the normal samples, activated NF-κB to at least about the same extent as MDP, demonstrating that the assay can differentiate the plasma of patients with a bacterial infection from the plasma of healthy donors.

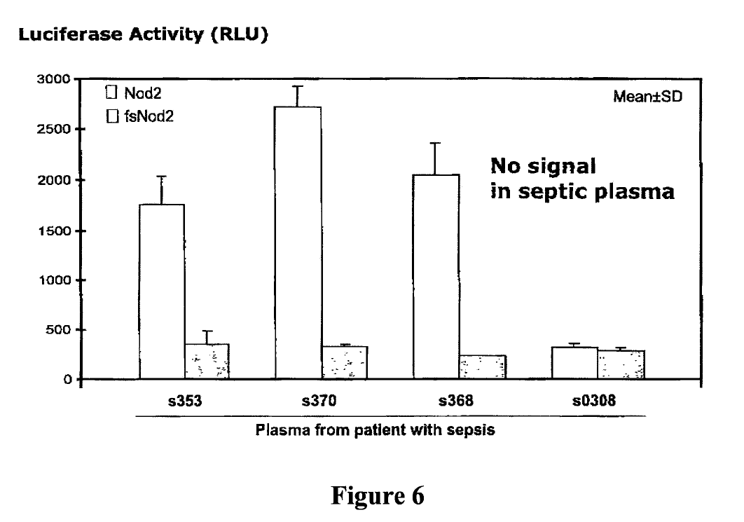

FIG. 6 demonstrates the specificity of the assay when detecting peptidoglycans from septic patients. Transfected NOD2, but not the frameshifted mutant, induced luciferase expression in cells co-transfected with pNF-κB-Luc.

Reporter assays of the invention can be standardized to detect PGN from aerobic bacteria, anaerobic bacteria, Gram-positive bacteria and Gram negative bacterial with defined sensitivities. The concentration of MPD or PGN in plasma can thus be extrapolated from the luciferase expression and a standard curve.

PGN activation can also be induced by bacterial translocation from the intestine of patients under certain conditions, such as abdominal surgery. However, the presence of PGN is transient (peak value occurs during the surgery) and disappears during the days following surgery. Our data suggest that, in contrast, PGN is detected in the plasma of septic patients longer than post-operative patients.

### Example 3

### NF-κB Activation Stimulated with Plasma from Surgical Patients

Human plasma samples from thirteen patients undergoing abdominal aortic surgery were obtained from the surgical team of Hôpital de la Pitié-Salpêtrière. Blood samples were taken prior to the induction of anesthesia, prior to incision, prior to clamping, following reperfusion, on day 1 post-surgery, and on day 2 post-surgery. In this precise clinical setting, each patient served as its own control.

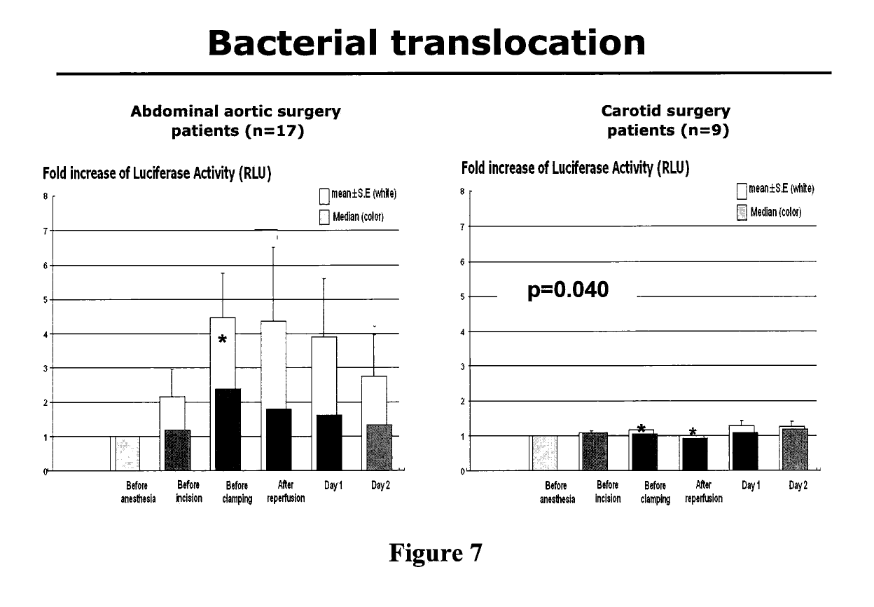

The bacterial translocation that occurs during surgery was detected by the luciferase assay of the invention. Luciferase expression began to rise following anesthesia and prior to incision; this increase may reflect an effect of anesthetic drugs on gastrointestinal motility and subsequent bacterial translocation. Luciferase expression rose further following incision and prior to aortic clamping, reflecting the bacterial translocation resulting from manipulation of the intestines by the surgical team. Luciferase expression decreased slowly following clamping, after reperfusion, and in the days following surgery. No PGN was detected in the plasma of patients undergoing carotid surgery. These patients are similar to abdominal aortic surgery patients in terms of clinical parameters and pathology, but during carotid surgery, there is no manipulation of the intestines (FIG. 7).

### Example 4

### NOD2 and TLR2 Increase Sensitivity of PGN Detection

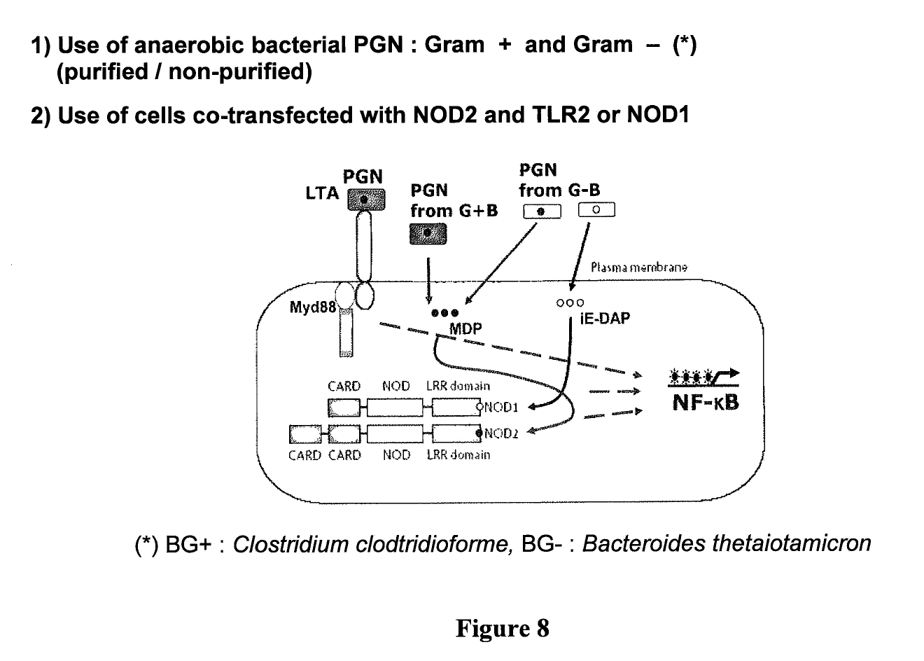

While highly purified peptidoglycan (PGN) have been claimed to only activate NOD2 (Travassos L H, Girardin S E, Philpott D J, et al. (2004) Toll-like receptor 2-dependent bacterial Sensing 65. does, not occur via peptidoglycan recognition. EMBO Rep, in press), this remains a controversial topic, and other studies identified TLR2 as another sensor (Dziarski R, Gupta D (2005) Staphylococcus aureus peptidoglycan is a toll-like receptor 2 activator: a reevaluation. Infect Immun, 73 5212-5216; Asong J, Wolfert M A. Maiti K, et al. (2009) Binding and cellular activation studies reveal that toll-like receptor 2 can differentially recognize peptidoglycan from gram-positive and -negative bacteria. J Biol Chem. (in press)). In addition, in “true life”, there is no highly purified circulating PGN. Thus, during infection, most probably, naturally released peptidoglycans are associated with lipoproteins, which are TLR2 ligands. A similar situation has been shown for the naturally released endotoxin by Hellman et al. (Hellman J, Roberts J D J, Tehan M M. et al. (2002)). Bacterial peptidoglycan-associated lipoprotein is released into the bloodstream in gram-negative sepsis and causes inflammation and death in mice (J Biol Chem, 277 14274-14280). Thus, both TLR2 and NOD2 can be involved in response to PGN released in vivo by bacteria. Accordingly, the addition of TLR2 improves the analysis of samples from patients to detect PGN and/or PGN-associated molecules, by increasing the sensitivity of the test. See FIG. 8.

More particularly, HEK cells at a density of 10⁵ cells/ml were co-transfected overnight with the NOD2 plasmid (1 ng) and TLR2 plasmid (100 ng) (Nahori M A, Fournié-Amazouz E, Que-Gewirth N S et al (2005), Differential TLR recognition of leptospiral lipid A and lipopolysaccharide in murine and human cells. J Immunol. 175. 6022-31), and the reporter gene under the control of NF-kappaB (75 ng) using 1% FuGENE 6 transfection reagent. A total plasmid concentration of 250 ng was achieved by addition of pcDNA 3.1 vector.

Anaerobic bacterial PGN (1-50 μg) were added to the transfected cells and incubated for 6 hours. Then, luciferase activity was assessed in cell lysates.

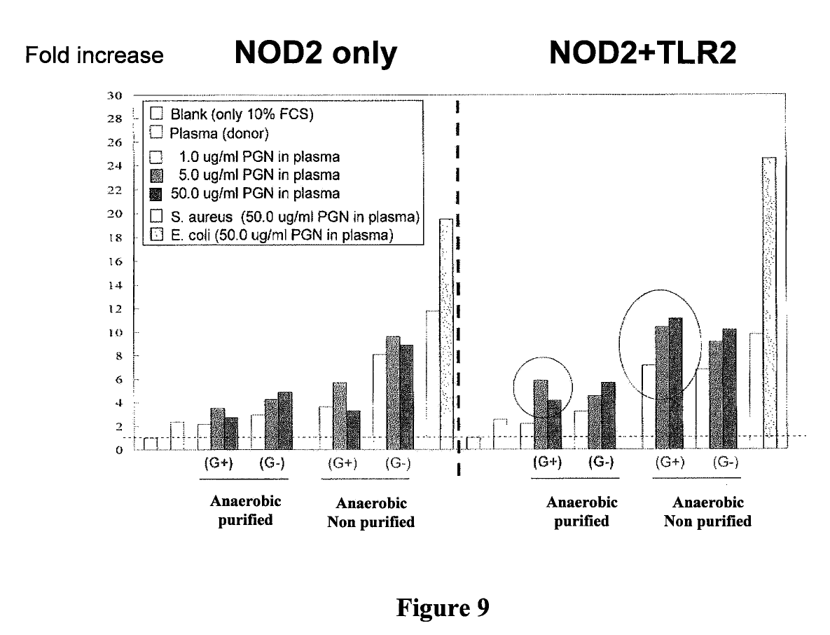

PGN preparations from Gram-positive anaerobic bacteria (Clostridium clostridioforme) added to human plasma from healthy donors allow a higher signal when cells are co-transfected with both NOD2 and TLR2 as compared to when only transfected with NOD2. FIG. 9.

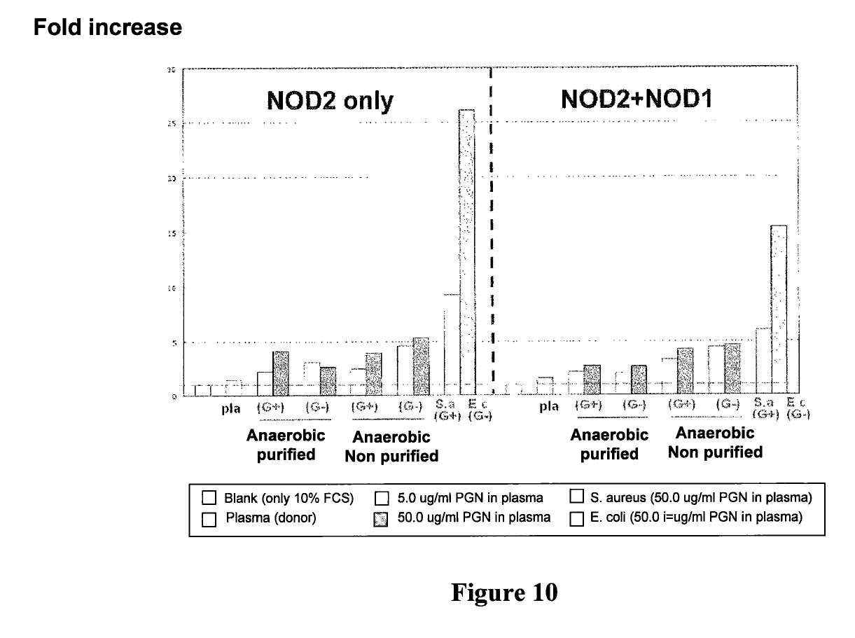

By comparison, the sensitivity of PGN detection was not improved by co-transfection of NOD2 with NOD1. FIG. 10.

In summary, co-transfection of NOD2 with TLR2 showed improved sensitivity of PGN detection. Increased responses were shown especially with anaerobic G+bacterial PGNs incubated in healthy plasma. In contrast, co-transfection of NOD2 with NOD1 did not show any improved sensitivity of PGN detection. Thus, in a preferred embodiment cells are not co-transfected with NOD2 and NOD1.

### Example 5

Endotoxins are only derived from Gram-negative bacteria and measuring peptidoglycan, which derives from both Gram-negative and Gram-positive bacteria may be more accurate. In patients undergoing abdominal aortic surgery, more than 90% of patients were positive for circulating peptidoglycan, indicating that microbial translocation can be more frequent than thought before. Furthermore, data indicates that the presence of peptidoglycan in the blood stream amplifies the level of the inflammatory response induced by surgical procedures.

Subjects and Operation

After approval of a study by the Ethics Committee for Human Research of Pitié-Salpêtrière Hospital (Session of Apr. 4, 2007), patients scheduled for abdominal aortic surgery (AAS) were included in this prospective observational study (n=21). As a control group, patients scheduled for carotid artery surgery (CAS) were also included (n=21). Excluded were patients undergoing coeloscopic surgery or surgery on the thoracic aorta, patients with signs of pre-operative infection, undergoing chronic dialysis, under anti-inflammatory medication or antibiotics treatment before surgery, presenting an on-going or neoplasic hematologic pathology, as well as immunodepressed patients. All patients gave informed consent.

The protocol followed for preoperative medication and anesthesia was similar in both groups of patients. The only difference was that treatment with anti-platelet aggregation agents was discontinued five days before surgery for AAS patients, whereas it was continued until the day of surgery for CAS patients. Usual premedication was maintained except converting enzyme inhibitors and angiotensin II antagonists, which were discontinued the day before surgery. All the patients were premedicated with midazolam 5 mg given orally 1 hour before surgery.

During operative period, all patients were anesthetized by target-controlled infusion (TCI) for propofol, sufentanil, and cisatracurium. Antibioprophylaxis was performed using cefamandole. Depending on hemodynamics and hematocrit, fluid loading was performed using crystalloid infusion (Ringer Lactate or isotonic saline) and colloid infusion (hydroxyethylstach 130/0.4), associated with blood transfusion if necessary to maintain hemoglobin level above 10 g/dl. Approximately 30 min before the end of surgery, all patients received paracetamol for postoperative analgesia, completed in recovery room with intravenous morphine until pain relief.

Blood Sampling

Blood samples were collected into the sodium citrated vacuum tubes as follows: immediately before anesthesia induction (T₁); before incision (T₂), before aortic clamping (AAS patients) or carotid artery clamping (CAS patients) (T₃) and after blood reperfusion (T₄) during the surgery, and on postoperative day one (POD1) and two (POD2) after the surgery. One 5 ml tube was immediately centrifuged and plasma samples were stored at −80° C. until assayed (within 2 months). Another 5 ml tube was used for the analysis of leukocyte-bound LPS.

Isolation of Peripheral Blood Mononuclear Cells (PBMC) from Whole Blood

For measurement of endotoxin associated with circulating leukocytes, PBMC were isolated from whole blood after dilution 1:1 with RPMI-1640 (Lonza, Verviers, Belgium) and centrifugation (680 g, 15° C. for 20 min) on Ficoll-Hypaque (Eurobio, France). After centrifugation, the cells at the medium/Ficoll interface were collected, washed with RPMI-1640 and centrifuged (350 g, 10° C. for 5 min). The pellet was resuspended with sterile endotoxin-free saline (0.9% NaCl) (Fresenius, France). PBMC were lysed by five cycles of freezing and thawing, and stored at −80° C. until assayed (within 2 months).

Detection of Circulating PGN in Human Plasma

Human embryonic kidney (HEK) 293T cells (ATCC, Manassas, Va.) were cultured in Dulbecco's modified Eagle's medium (Invitrogen, Carlsbad, Calif.) supplemented with 10% fetal calf serum (PAA, Pasching, Austria). The detection of PGN in biological fluids using this transfected cell line has been described in U.S. Provisional Application No. 61/064,633. Briefly, HEK293T cells were transfected with a plasmid permitting the constitutive expression of NOD2 (sensor of both Gram-positive and Gram-negative PGN) and an NF-κB-dependent reporter gene coding for luciferase. Fifty μl of plasma were added to the transfected cells and incubated for 6 hours at 37° C. The presence of PGN in plasma was assessed by luciferase activity in cell lysates. The results from study subjects were presented as fold change of luciferase activity based on the initial values (T₁, before anesthesia). The detection limit was calculated based on the mean value±2 standard deviations (SD) of the fold values at T₂ in control subjects. Each test was performed in triplicate. A fold increase of 1.57 was considered to be positive.

Measurement of Endotoxin in Peripheral Blood Mononuclear Cells (PMBC) and Plasma

Detection of bacterial endotoxin in plasma is hindered by the yellow color of plasma and the presence of inhibitors. The background absorbance of plasma was avoided by using a diazo-coupling Limulus amebocyte lysate (LAL) assay that gives a magenta coloration (detection limit: 1.6 pg/ml) (Associates of Cape Cod, East Falmouth, Mass.), and by heating plasma at 65° C. for 30 min to inactivate inhibitors as described by Adrie C, Adib-Conquy M, Laurent I, Monchi M, Vinsonneau C, Fitting C, Fraisse F, Dinh-Xuan A T, Carli P, Spauling C, et al. Successful cardiopulmonary resuscitation after cardiac arrest as a “Sepsis-like” Syndrome. Circulation 2002; 106:562-568. Endotoxins from lysates of PBMC were assayed with QCL-1000 Chromogenic LAL kit (detection limit: 5 pg/ml) (Lonza, Walkersville, Minn.). The assays were carried out according to the manufacturers' instructions. Samples were tested in duplicate.

Detection of Bacterial PGN in Plasma Samples from AAS and CAS Patients

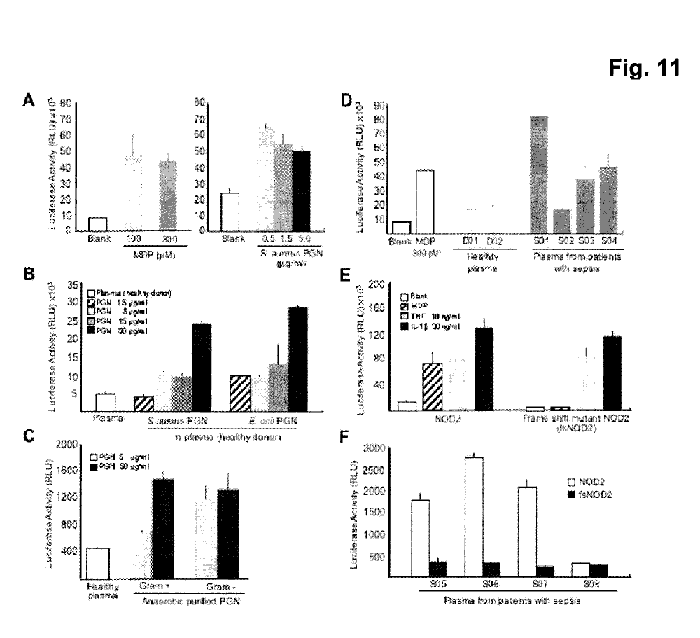

Bacterial PGN was measured in patients' plasma samples by the test according to the invention. This in vitro test was able to detect specifically PGN from Gram-positive/Gram-negative, aerobic/anaerobic bacteria. Controls for specificity and sensitivity of this test can be found in FIG. 11.

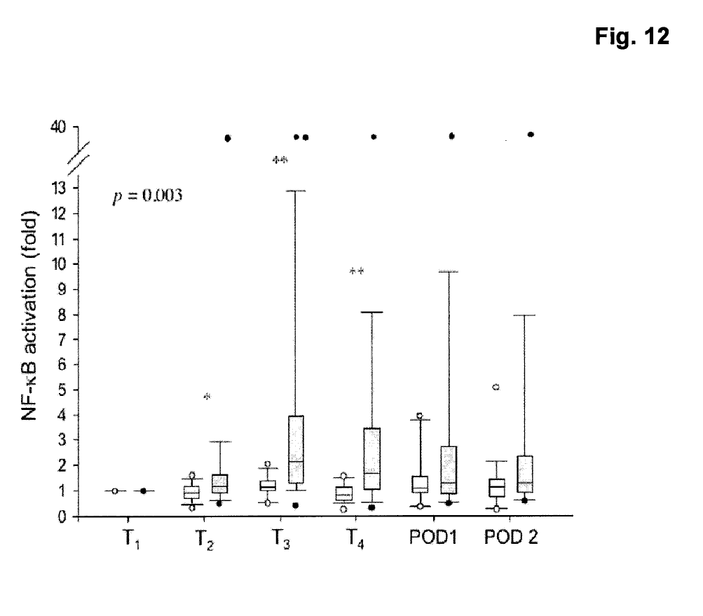

FIG. 12 presents the results for the detection of circulating PGN during and after surgery in the two groups. Values are provided as fold change of luciferase activity as compared to NF-κB activation before anesthesia (T₁). PGN was detected in the plasma of 90.5% of AAS patients, with a peak occurring before aortic clamping (T₃). PGN levels were still high after blood reperfusion (T₄), and then gradually declined at POD1 and POD2. In contrast, no major increase occurred in the plasma of CAS patients. PGN was detected in the plasma of 23.8% of these patients during surgery, but the levels were significantly lower than in AAS patients. One AAS patient, who developed SIRS at POD1, had very high levels of PGN (20˜40 fold increase of NF-κB activation) during the whole observational period starting at time point T₂, which led to a statistically significant difference in the values between the two groups at T₂. Before surgery, this patient already had large and numerous calcified atheromas in the aorta and other arteries throughout the body, which may be responsible for increased vascular fragility and/or increased ischemia, even before gut manipulation and clamping. If this patient was removed from the analysis, there would be no significant differences between the two groups at time point T₂, but there were still significant differences in NF-κB activation between AAS and CAS patients before aortic clamping (T₃) and after blood reperfusion (T₄).

Endotoxin Levels Bound to PBMC and in Plasma

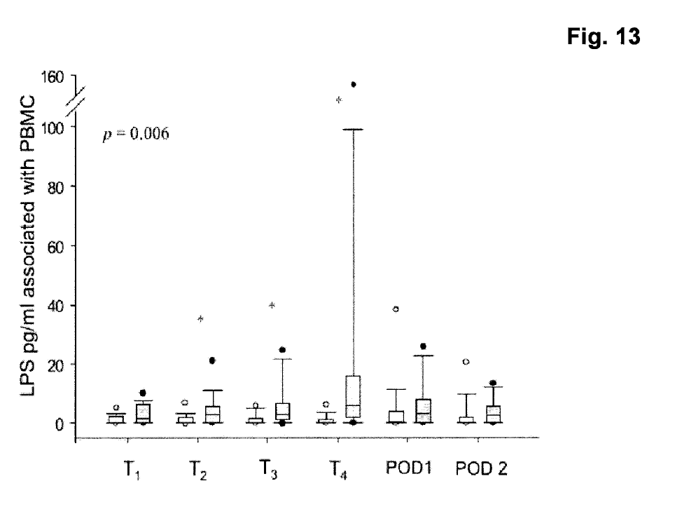

The levels of endotoxin released from lysates of PBMC are presented in FIG. 13. LPS was detected in PBMC lysates of 71.4% of AAS patients, with levels significantly higher than in CAS patients. Highest levels of LPS were observed after blood reperfusion (T₄) in AAS patients and decreased progressively at POD1 and POD2. On the other hand, the CAS group showed no significant presence of LPS on their PBMC during the observational period. Plasma levels of LPS were also measured in AAS patients. At T₄, the median value was 1.7 pg/ml [range: 0-11.5 pg/ml]; the mean was 2.7±0.7 pg/ml, and was significantly higher than the levels measured at T₁ (p<0.007).

Comparison of the Sensitivities of the Assays for the Detection of PGN and Endotoxin

In order to examine the sensitivity of detection of the PGN and endotoxin tests, the positive responses to the tests based on the detection limits were compared. The detection limits of the LAL test in PBMC and plasma were 5 pg/ml and 1.6 pg/ml, respectively. Because the natural structure derived from PGN following translocation is unknown and may be different from that resulting from enzymatic digestion and purification performed in vitro, extrapolating the levels of luciferase activity to a standard curve of biochemically purified PGN was avoided. The test was expressed as fold increase as compared to the plasma before surgery, each patient being his own control. The detection limit of the PGN test was 1.57 fold increase in luciferase activity as compared to the value at T₁ (before anesthesia). This detection limit was calculated on the basis of the mean±2SD of the fold increase at T₂ in control patients. For reference, the mean±SEM and median fold increase for various PGN (5.0 μg/ml) incubated in healthy plasma were 1.76±0.19 and 1.68, respectively.

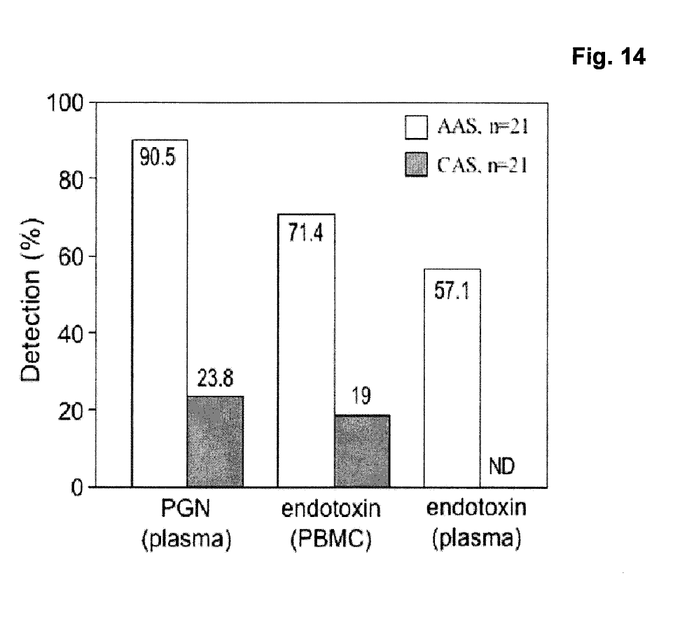

In all AAS and CAS patients, the detection sensitivity for the PGN test in plasma and the endotoxin test in PBMC were compared. In AAS patients, 90.5% (19 out of 21 patients) were positive in the PGN test, 71.4% (15 out of 21 patients) were positive in the LAL test used to assess PBMC-associated LPS, and 57.1% ( 12/21) had measurable circulating LPS (FIG. 14). In CAS patients, 23.8% (5 out of 21 patients) were positive in the PGN test, and 19% (4 out of 21 patients) in the LAL test done to measure PBMC-associated LPS. From this result it was concluded that the PGN detection test was more sensitive than any endotoxin tests.

Derangement in gut barrier function occurs in many clinical settings. Endotoxin translocation has been evidenced in some cases and more frequently than systemic bacterial translocation. Still, endotoxin being only representative of Gram-negative bacteria, microbial translocation may occur more regularly than previously reported after endotoxin measurement. Aims of this invention were (i) to develop a new tool allowing one to measure peptidoglycan, representative of both Gram-negative and Gam-positive; (ii) to address its presence within the blood stream of patients in a clinical situation known to favor translocation of microbial products; and (iii) to compare with endotoxin measurements.

Abdominal aortic surgery (AAS) is thought to be associated with endotoxin and bacterial translocation through the gut barrier, following manipulation of the gut by the surgeon and aortic clamping. However, evidence to prove this link was difficult to gather, probably because bacteria translocated into the bloodstream are rapidly killed and do not give rise to positive hemocultures. Shimizu T, Tani T, Endo Y, Hanasawa K, Tsuchiya M, Kodama M. Elevation of plasma peptidoglycan and peripheral blood neutrophil activation during hemorrhagic shock: Plasma peptidoglycan reflects bacterial translocation and may affect neutrophil activation. Crit Care Med 2002; 3:0:77-82. Alexander J W. Gianotii L, Pyles T, Carey M A. Babcock G F. Distribution and survival of Escherichia coli translocating from the intestine after thermal injury. Ann Surg 1991; 213:558-566:discussion 566-557. Several studies aimed to address this question by measuring circulating endotoxin. Adrie C, Adib-Conquy M, Laurent I, Monchi M, Vinsonneau C, Fitting C, Fraisse F, Dinh-Xuan A T, Carli P, Spauling C, et al. Successful cardiopulmonary resuscitation after cardiac arrest as a “Sepsis-like” Syndrome. Circulation 2002; 106:562-568. Cabié A, Parkas J-C, Fitting C, Laurian C, Cormier J-M, Carlet J, Cavaillon J-M. High levels of portal TNFα during abdominal aortic surgery in man. Cytokine 1993; 5:448-453. However, this approach is hindered by the presence of many interfering or blocking molecules (soluble CD14 LPS-binding protein, lipoproteins). Tapping R I, Tobias P S. Cellular binding of soluble CD 14 requires lipopolysaccharide (LPS) and LPS-binding protein. J Biol Chem 1997:272:23157-23164. Lamping N, Dettmer R, Schroder N W, Pfeil D, Hallatschek W, Burger R, Schumann R R. LPS-binding protein protects mice from septic shock caused by LPS or Gram-negative bacteria. J Clin Invest 1998; 101:2065-2071. Cavaillon J M, Fitting C. Haeffner-Cavaillon N, Kirsch S J, Warren H S. Cytokine response by monocytes and macrophages to free and lipoprotein-bound lipopolysaccharide. Infect Immun 1990; 58:2375-2382. Thus, the determination of LPS levels in the plasma of patients after surgery was not reliable enough, and did not allow to demonstrate that translocation was taking place systematically.

This invention provides a new method for detecting bacterial PGN in plasma, using a cell line transfected with NOD2, a general sensor of PGN through its minimal motif MDP, Inohara N, Ogura Y, Fontalba A, Gutierrez O, Pons F, Crespo J, Fukasc K, Inamura S, Kusumoto S, Hashimoto M, et al. Host recognition of bacterial muramyl dipeptide mediated through NOD2. Implications for Crohn's disease. J Biol Chem 2003; 278:5509-5512. Girardin S E, Boneca I G, Viala J, Chamaillard M, Labigne A, Thomas G, Philpott D J, Sansonetfi P J, NOD2 is a general sensor of peptidoglycan through muramyl dipeptide (MDP) detection. J Biol Chem 2003:278:8869-8872 and a reporter gene under the control of the NF-κB transcription factor. Measurement of PGN is relevant in many aspects; first, it is the major component of Gram-positive bacteria and is also found in Gram-negative bacterial. Thus, while LPS detection addresses only Gram-negative bacteria, PGN detection addresses both types. Second, the system of the invention efficiently detected anaerobic bacterial PGN, expected to be more representative of the intestinal flora. Finally, the specificity of the system of the invention was confirmed by the use of a frameshift mutant of NOD2, which cannot be activated by bacterial PGN or its fragment. Inamura S, Kusumoto S, Hashimoto M, et al. Host recognition of bacterial muramyl dipeptide mediated through NOD2. Implications for Crohn's disease. J Biol Chem 2003; 278:5509-5512. Girardin S E, Boneca I G, Viala J, Chamaillard M, Labigne A, Thomas G, Philpott D J, Sansonetfi P J, NOD2 is a general sensor of peptidoglycan through muramyl dipeptide (MDP) detection. J Biol Chem 2003:278:8869-8872. This system showed that translocation of pathogen-associated molecular patterns (PAMPs) occurred with a high frequency in abdominal aortic surgery, and that the assay of PGN in plasma is an useful and informative tool for early detection of a bacterial presence in the blood stream. This is the first time that PGN was measured and found in AAS patients. Its detection is more sensitive than that of LPS, even if one could enhance the sensitivity of the detection of endotoxin by measuring that associated with PBMC, in agreement with a previous study performed with five Neisseria meningitidis-infected patients. The results of this invention concur with those of a hemorrhagic shock study in rat, showing that 30% had detectable amounts of LPS, but 73% were positive for circulating PGN. Shimizu T, Tani T, Endo Y, Hanasawa K, Tsuchiya M, Kodama M. Elevation of plasma peptidoglycan and peripheral blood neutrophil activation during hemorrhagic shock: Plasma peptidoglycan reflects bacterial translocation and may affect neutrophil activation. Crit Care Med 2002; 3:0:77-82.

On the other hand, CAS patients (negative control) did not show peaks for circulating PGN or endotoxin, even if in some rare cases, PGN was detected in their plasma. Of course the CAS group underwent a shorter and less severe insult (shorter duration of surgery, less blood loss, rare blood transfusion). Nevertheless, CAS patients were relevant as a control group for AAS patients. The groups were comparable in terms of weight, sex ratio and pathology prior to surgery (atherosclerosis, diabetes, smocking habits). They were also undergoing vascular surgery, but without intestinal manipulation for CAS patients. Bacterial translocation is indeed tightly linked to gut manipulation during surgery.

Regarding medications, especially for statin, the CAS were higher consumers than AAS patients even though the difference was not statistically significant. Statins are known to have pleiotropic effects such as reduction in inflammatory response, stabilization of atherosclerotic plaques and improvement in vascular endothelial function, besides the lipid lowering effect. Statin consumption had no significant effect on the levels of PGN. These observations suggest that differences in circulating PGN between the two surgery groups during the observational period were not related to statin consumption.

Set-Up of the PGN Detection System

To develop a new tool for the detection of circulating PGN, HEK293T cell line was used constitutively expressing NOD2, as sensor of bacterial PGN through its minimal motif MDP. Girardin S E, Boneca I G, Viala J, Chamaillard M, Labigne A, Thomas G, Philpott D J, Sansonetti P J. NOD2 is a general sensor of peptidoglycan through muramyl dipeptide (MDP) detection. J Biol Chem 2003; 278:8869-8872. Inohara N, Ogura Y, Fontalba A, Gutierrez O, Pons F, Crespo J, Fukase K, Inamura S, Kusumoto S, Hashimoto M, et al. Host recognition of bacterial muramyl dipeptide mediated through NOD2. Implications for crohn's disease. J Biol Chem 2003; 278:5509-5512. The cell line was transfected with a luciferase reporter gene under the control of an NF-κB-dependent promoter. Id.

This system was able to give positive signals when simulated with MDP (FIG. 11, left panel) or PGN from S. aureus (FIG. 11A, right panel). The detection system was also functional when PGN was added to healthy donor's plasma (FIG. 11B).

The system efficiently detected various concentrations of PGN from S. aureus (Gram-positive bacteria) or E. coli (Gram-negative bacteria) incubated in plasma from healthy donors. Gut flora is mainly composed of anaerobic bacteria, and the detection of anaerobic bacterial PGN may be more relevant for bacterial translocation. Thus, the system was checked to determine whether it was responsive to the stimulation with purified Gram-positive as well as Gram-negative anaerobic bacterial PGN incubated in healthy plasma (FIG. 11C).

NF-κB activation was also obtained with plasma samples from sepsis patients, used as positive controls, as compared to those from healthy donors (FIG. 11D).

The specificity of the system was checked by transfecting an expression plasmid for frameshift NOD2 (fsNOD2), a mutant unable to activate NF-κB in response to MDP (FIG. 11E). Cell lines transfected with NOD2 or fsNOD2 were similarly responsive to inflammatory cytokines, such as TNF-α and IL-1β. Plasma samples from sepsis patients, which showed a positive signal in the NOD2 transfected system did not induce activation in the fsNOD2 transfected system (FIG. 11F). These results indicate the NOD2 transfection system can selectively and specifically detect bacterial PGN products.

In summary, the gut is often considered as the motor of critical illness through bacterial translocation, which amplifies the inflammatory response and alters the immune status. However, systemic bacterial translocation was rarely proven and endotoxin measurement only reflects translocation of Gram-negative-derived products. The process could be more frequently identified if peptidoglycan, derived from both Gram-negative and Gram-positive bacteria was measured. This invention provides a new tool to detect circulating peptidoglycan using a NOD2-transfected cell line. Patients undergoing abdominal aortic surgery (AAS) (n=21) were studied. Patients undergoing carotid artery surgery (CAS) (n=21) were included as a negative control group. In 90.5% of the AAS patients, a peptidoglycan peak was detected in plasma before aortic clamping and persisted after blood reperfusion. As expected, no peak was detected in plasma from CAS patients (P=0.003). Regarding leukocyte-bound endotoxin levels, a peak appeared after blood reperfusion in AAS patients, but not in CAS patients (P=0.006). Levels of interleukin (IL)-6, IL-10, C-reactive protein, and procalcitonin were maximal at postoperative day 1 or 2 in AAS patients. The levels of circulating peptidoglycan after reperfusion positively correlated with those of CRP on day 1 post-AAS (P<0.001). The measurement of circulating peptidoglycan gives a highly informative tool for early and sensitive detection of bacterial translocation.

Endotoxins are only derived from Gram-negative bacteria and measuring peptidoglycan, which derives from both Gram-negative and Gram-positive bacteria could be more accurate. This invention also shows that in patients undergoing abdominal aortic surgery, most patients were positive for circulating peptidoglycan, indicating that microbial translocation can be more frequent than thought before.

### REFERENCES

The specification is most thoroughly understood in light of the following references, all of which are hereby incorporated in their entireties. The disclosures of the patents and other references cited above are also hereby incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.
Adib-Conquy M, Monchi M, Goulenok C, Laurent I, Thuong M, Cavaillon J-M, Adrie C (2007) Enhanced plasma levels of soluble triggering expressed on myeloid cells-1 and procalcitonin after cardiac surgery and cardiac arrest in the absence of Infection, Shock 28:406-10.
Adrie C, Adib-Conquy M, Laurent I, Monchi M, Vinsonneau C, Fitting C, Fraisse F, Dinh-Xuan A T, Carli P, Spaulding C, Dhainaut J-F, Cavaillon J-M (2002) Successful cardiopulmonary resuscitation after cardiac arrest as a “sepsis-like” syndrome, Circulation 106:562-8.
Begue B, Dumant C, Bambou J C, Beaulieu J F, Chamaillard M, Hugot J P, Goulet O, Schmitz J, Philpott D J, Cerf-Bensussan N, Ruemmele F M (2006) Microbial induction of CARD15 expression in intestinal epithelial cells via toll-like receptor 5 triggers an antibacterial response loop, J. Cell. Physiol. 209:241-252.
Balzan S, de Almeida Quatros C, de Cleva R, Zilberstein B, Cecconello I (2007) Bacterial translocation: overview of mechanisms and clinical impact, J. Gastroenterol. Hepatol. 22:464-71.
Cabiè A, Farkas J C, Fitting C, Laurian C, Cormier J M, Carlet J, Cavaillon J-M (1993) High levels of portal TNF-alpha during abdominal aortic surgery in man, Cytokine 5:448-53.
Chamaillard M, Philpott D, Giardin S, Zovali H, Lesage S, Chareyre F, Bui T H, Giovannini M, Zaehringer U, Penard-Lacronique V, Sansonetti P I, Hugot J P, Thomas G (2003) Proc. Natl. Acad. Sci USA 100:3455-3460.
Dziarski R, Gupta D (2005) Staphylococcus aureus peptidoglycan is a toll-like receptor 2 activator: a reevaluation, Infect Immun. 73:5212-6.
Girardin S E, Boneca I G, Carneiro L A M, et al. (2003a) NOD1 detects a unique muropeptide from Gram-negative bacterial peptidoglycan, Science 300:1584-1587.
Girardin S E, Boneca I G, Viala J, et al. (2003b) NOD2 is a general sensor of peptidoglycan through muramyl dipeptide (MDP) detection, J. Biol. Chem. 278:8869-8872.
Inohara N, Koseki T, Lin J, del Peso L, Lucas P C, Chen F F, Ogura Y, Nuñez G (2000) An induced proximity model for NF-κB activation in the NOD1/RICK and RIP signaling pathways, J. Biol. Chem. 275:27823-27831.
Park J-H, Kim Y-G, McDonald C, Kanneganti T-D, Hasegawa M, Body-Malapel M, Inohara N, Núñez G (2007) RICK/RIP2 mediates innate immune responses induced through Nod1 and Nod2 but not TLRs, J. Immunol. 178:2380-2386.
Shimizu T, Endo Y, Tabata T, Mori T, Hanasawa K, Tsuchiya M, and Tani T (2005) Diagnostic and predictive value of silkworm larvae plasma test for postoperative infection following gastrointestinal surgery, Crit. Care Med. 33:1288-95.
Tanabe T, Chamaillard M, Ogura Y, Zhu L, Qiu S, Masumoto J, Ghosh P, Moran A, Predergast M M, Tromp G, Williams C J, Inohara N, Núñez G (2004) Regulatory regions and critical residues of NOD2 involved in muramyl dipeptide recognition, EMBO J. 23:1587-1597.
Tang B M P, Eslick G D, Craig J C, McLean ASM (2007) Accuracy of procalcitonin for sepsis diagnosis in critically ill patients: systematic review and meta-analysis, Lancet Infect Dis. 7:210-217.

Travassos L H, Girardin S E, Philpott D J, Blanot D, Nahori M A, Werts C, Boneca I G (2004) Toll-like receptor 2-dependent bacterial sensing does not occur via peptidoglycan recognition, EMBO Rep. 5:1000-1006.
Voss E, Wehkamp K, Stange E F, Schröder J M, Harder J (2006) NOD2/CARD15 mediates induction of the antimicrobial peptide human beta-defensin-2. J. Biol. Chem. 281:2005-2011.

1. A method for detecting a peptidoglycan or a peptidoglycan-like compound in a sample comprising
(a) transfecting a cell with a vector comprising a nucleotide sequence encoding NOD2 or nucleotide sequences encoding NOD2 and TLR2;
(b) co-transfecting said cell with a vector comprising a nucleotide sequence encoding a reporter protein;
(c) placing said cell in contact with the sample; and
(d) measuring the reporter gene expression in said cell,
wherein the reporter gene expression indicates the presence of peptidoglycan or peptidoglycan-like compound in the sample.

2. The method as claimed in claim 1, wherein peptidoglycan is detected by measuring activation of a transcription factor activated by NOD2.

3. The method as claimed in claim 2, wherein the transcription factor is NF-κB.

4. The method as claimed in claim 1, wherein the reporter gene is under the control of a promoter that contains transcription recognition sequences for the binding of a transcriptional factor activated by NOD2.

5. The method as claimed in claim 4, wherein the vector comprising the sequence encoding the reporter protein further comprises an enhancer region of the transcription factor.

6. The method as claimed in claim 4, wherein the transcription factor is NF-κB.

7. The method as claimed in claim 1, wherein the reporter gene is detected by a bioluminescent signal.

8. The method as claimed in claim 1, wherein the sample is plasma.

9. The method as claimed in claim 1, wherein the sample is taken from a patient with sepsis.

10. The method as claimed in claim 1, wherein the sample is taken from a surgical patient.

11. The method as claimed in claim 8, wherein the sample is taken prior to, during, or after surgery.

12. The method as claimed in claim 8, wherein the surgery comprises abdominal aortic surgery.

13. The method of claim 1, wherein NOD2 is human NOD2.

14. The method of claim 1, wherein the transfection is transient.

15. A method for detecting the presence or absence of a bacterial infection in a sample, wherein the method consists essentially of:
(a) providing a sample from a subject;
(b) providing a cell having an intact cell membrane, and wherein the cell expresses NOD2 or NOD2 and TLR2 and a reporter gene under the control of a promoter that contains transcription recognition sequences for the binding for a transcriptional factor activated by NOD2;
(c) contacting the cell and the sample by extracellular contact of the sample with the intact wall of the cell and without injecting the sample into the cell;
(d) incubating the cell from (c) under conditions to maintain viability of the cell;
(e) determining whether the reporter gene is expressed; and
(f) correlating expression of the reporter gene with the presence or absence of the bacterial infection.

16. The method as claimed in claim 15, wherein the extracellular contact occurs in the absence of cell transfection reagents.

17. The method as claimed in claim 15, wherein the extracellular contact occurs in the absence of lipid emulsifying or solubilizing compounds.

18. The method as claimed in claim 15, wherein the sample is plasma.

19. The method as claimed in claim 18, wherein the sample is plasma from a patient undergoing surgery.

20. The method as claimed in claim 15, wherein the cell is a human embryonic kidney cell and the reporter is luciferase.

21. A kit for detecting peptidoglycan and peptidoglycan-like compounds according to the method of claim 1 comprising:
(a) a vector comprising a sequence encoding NOD2 or NOD2 and TLR2;
(b) a vector comprising a sequence encoding a reporter protein;
(c) cells to be transfected with said vectors; and
(d) instructions for use.

22. The kit as claimed in claim 21, whereas the sequence coding for the reporter protein is under the control of a promoter that contains transcription recognition sequences for the binding of a transcription factor that is activated by NOD2.
